(19) United States
(12) Patent Application Publication
(10) Pub. No.: US 2026/0363885 A1
(43) Pub. Date: Nov. 19, 2026

(54) STENT DELIVERY SYSTEM, STENT WITH TRACTION THREAD USED THEREFOR, AND INNER CATHETER USED THEREFOR

(71) Applicant: BCM Co., LTD., Gyeonggi-do (KR)

(72) Inventors: Byung Cheol MYUNG, Gyeonggi-do (KR); Oh Cheon KWON, Gyeonggi-do (KR)

(21) Appl. No.: 19/267,679

(22) Filed: Jul. 14, 2025

(30) Foreign Application Priority Data
May 16, 2025 (KR) 10-2025-0063732

(51) Int. Cl. A61F 2/95; A61F 2/966

(52) U.S. Cl. CPC A61F 2/9517; A61F 2/966; A61F 2002/9665

(57) ABSTRACT

Disclosed relates to a stent delivery system, a stent with a traction thread used therefor, and an inner catheter used therefor, wherein the stent delivery system is capable of facilitating movement of an outer catheter in a lumen and passage thereof in a narrow lesion by preventing the thickness of a portion of the outer catheter from becoming thicker within the length of the traction thread, and the traction thread of a single strand is woven around all space parts closest to an end of the stent, and then protrudes outwards of the stent at opposite parts of the traction thread having different lengths, and the opposite parts having different lengths are tied together with a first knot part formed close to the end of the stent and one end of the traction thread protruding from the first knot part is tied to form a second knot part.

### CROSS REFERENCE TO RELATED APPLICATION

The present application claims priority to Korean Patent Application No. 10-2025-0063732, filed May 16, 2025, the entire contents of which are incorporated herein for all purposes by this reference.

### BACKGROUND OF THE INVENTION

### Field of the Invention

The present disclosure relates to a stent delivery system, a stent with a traction thread used therefor, and an inner catheter used therefor, the stent delivery system being used to place the stent into a lesion area to expand a stenotic or occlusive lesion area occurring in a lumen of the human body to maintain a passage in the lumen.

### Description of the Related Art

In general, when a stenotic or occlusive lesion occurs in a lumen of the body, such as the esophagus, duodenum, biliary tract of the human body, and the like, its own function to move fluid is decreased.

Therefore, conventionally, a stent is inserted into the location of the stenotic or occlusive lesion area to expand the narrowed lumen.

In this respect, in patent document 1, there is provided a hanger loop structure of a traction thread for removing a stent, with the traction thread provided to remove the stent including space parts formed by weaving or crossing one or more strands of shape memory alloy wires into a zigzag shape and a cylindrical body with a plurality of bent ends formed at opposite ends in the circumference thereof, and the hanger loop structure is configured such that the traction thread is woven to zigzag around the bent ends of the cylindrical body of the stent into a circular strip shape and then opposite ends of the circumferential traction thread protrude long and then are tied up together to form a hanger loop.

However, in the patent document 1, as shown in FIGS. 1 and 2, when the stent is compressed by an outer catheter 2a of the stent delivery system and mounted into a mounting space 2b′ of an inner catheter, two parts of traction threads are located and overlapped between the outer catheter and the stent and between the outer catheter and the inner catheter, which increase the thickness of a portion of the outer catheter within a length of the two parts of the traction thread.

For this reason, the thickened outer catheter may be difficult to be moved due to resistance in the lumen, or may not easily pass through a narrow lesion.

### DOCUMENT OF RELATED ART

### Patent Document

Patent Document 1: Korean Patent Application Publication No. 10-2010-0119068 (date of publication: Nov. 9, 2010)

### SUMMARY OF THE INVENTION

Accordingly, an objective of the present disclosure is to provide a stent delivery system, a stent with a traction thread used therefor, and an inner catheter used therefor, the stent delivery system being capable of facilitating movement of an outer catheter in a lumen and passing thereof through a narrow lesion as the thickness of a portion of the outer catheter is prevented from becoming thicker within the length of the traction thread.

In order to achieve the above objective, according to the present disclosure, there is provided a stent with a traction thread, wherein the traction thread may be made up of a single strand, and the traction thread may be woven around all space parts closest to an end of the stent, and then protrude outwards of the stent at opposite parts of the traction thread having different lengths, and the opposite parts having different lengths of the traction thread may be tied together with a first knot part formed close to the end of the stent and one end of the traction thread protruding from the first knot part may be tied to form a second knot part.

Furthermore, according to the present disclosure, there is provided an inner catheter that may be movably inserted into an outer catheter of a stent delivery system, and connected to a guide tip caught by an opening of the outer catheter at one portion thereof, and include a mounting space formed close to the guide tip, in which a stent with a traction thread at one portion thereof may be compressed by the outer catheter and mounted, wherein one portion of the traction thread protruding from the stent may be tied to form a second knot part, and a ring-shaped first inserting groove may be formed behind the mounting space of the inner catheter so that the second knot part may be inserted into the first inserting groove.

Furthermore, according to the present disclosure, there is provided a stent delivery system including: a first handle connected to an outer catheter moved along a lumen of human body, a second handle connected to a guide tube movably inserted into the first handle and the outer catheter, and an inner catheter, which is connected to the guide tube and movably inserted into the outer catheter and includes one portion connected to a guide tip caught by an opening of the outer catheter, and, at the portion, includes a mounting space, in which a stent with a traction thread is compressed by the outer catheter and mounted, formed close to the guide tip, wherein the stent may be placed into the lesion area in order to expand a stenotic or occlusive lesion area occurring in the lumen and maintain a passage in the lumen.

According to the present disclosure, a single strand of the traction thread, not a pair of strands, is located and overlapped between the outer catheter and the stent and between the outer catheter and the inner catheter, so that the thickness of the portion of the outer catheter where the traction thread is overlapped does not become thicker.

In other words, the thickness of a portion of the outer catheter does not become thicker within the length of the traction thread.

For this reason, unlike the conventional stent delivery system, the outer catheter of the present disclosure that is not thick can be easily moved with reduced resistance in the lumen during stent placement with the stent delivery system, and can pass through a narrow lesion easily.

Furthermore, since the outer catheter having a large diameter for reducing resistance by the traction thread is not required, it is possible to prevent the outer catheter of an increased diameter from being moved in the lumen and passing through the narrow lesion with difficulty.

When the outer catheter is pulled from the lesion to unfold the stent, the second knot part of the traction thread inserted into the first inserting groove of the inner catheter is not caught by an inner surface of the outer catheter, so that the outer catheter can be pulled well without resistance and the stent placement can be efficiently performed.

Furthermore, the outer catheter does not need to be pulled with a greater external force, preventing the outer catheter from being torn or cut due to the greater external force.

When a certain amount of time has passed after the stent is implanted into the lesion, the stent can be easily removed by pulling the second knot part with a pincer tool, such as forceps.

For this reason, pincer tool, such as forceps may prevent the outer catheter from slipping off the traction thread.

The first inserting groove has a ring shape, and a portion of the inner catheter with reduced thickness can be supported by the support ring inserted into the first inserting groove.

Furthermore, the both side portions of the support ring are inserted into the second inserting grooves, so that a supporting area of the support ring is increased and the supporting force can be increased.

For this reason, when the outer catheter and the guide tip are moved, it is possible to prevent the inner catheter from being easily bent or bent and cut when caught by the lumen and a lesion.

The distance is formed between an end of the stent and the first knot part, when the compressed stent extends, it is possible to prevent the opening of the stent from not expanding to the original state when caught by the first knot part.

For this reason, the stent can expand a portion of the lesion efficiently.

The first inserting groove is formed in a long shape along the circumference of the inner catheter, and the first inserting groove does not need to have a ring shape in the inner catheter.

In other words, as the first inserting groove is formed, it is possible to prevent the thickness of a portion of the inner catheter from being thin as much as possible.

For this reason, this can prevent the inner catheter from being easily bent when caught by the lumen and the lesion, without the support ring.

The angle between the opposite ends of the first inserting groove in the circumferential direction of the inner catheter is formed to range from 90° to 180°, so that the second knot part can be easily inserted into the first inserting groove, and a portion of a holding force of the inner catheter with the first inserting groove can be prevented from being weakened when caught by the lumen and the lesion.

### BRIEF DESCRIPTION OF THE DRAWINGS

FIGS. 1 and 2 are a sectional view and a partially enlarged view showing an operational state of a conventional stent delivery system,

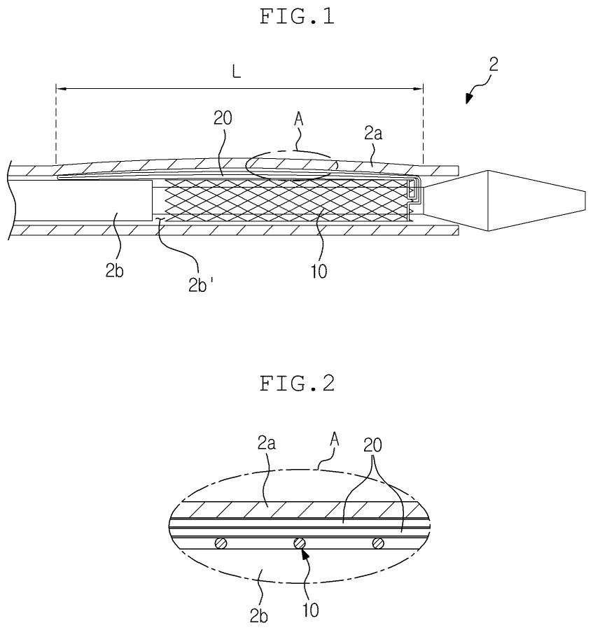

FIG. 3 is a front view showing a stent delivery system according to an embodiment of the present disclosure to which a stent with a traction thread and an inner catheter are used,

FIGS. 4 to 6 are partially sectional views of FIG. 3,

FIGS. 7 and 8 are partially enlarged views of FIG. 4,

FIG. 9 is a front view of the stent with the traction thread according to the embodiment of the present disclosure,

FIGS. 10 and 11 are partially enlarged views of FIG. 9,

FIG. 12 is an operational view showing a reason of forming a distance between the stent and the first knot part according to the embodiment of the present disclosure,

FIG. 13 is a perspective view showing an inner catheter according to the embodiment of the present disclosure,

FIG. 14 is a sectional line taken along line A-A of FIG. 13,

FIGS. 15 to 18 are operational views showing the stent delivery system with the stent with the traction thread and the inner catheter according to the embodiment of the present disclosure are used,

FIGS. 19 and 20 are sectional views showing the inner catheter according to a modified example of the embodiment of the present disclosure.

### DETAILED DESCRIPTION OF THE DISCLOSURE

Accordingly, exemplary embodiments of the present disclosure will be described in detail will be described with reference to the accompanying drawings as follows.

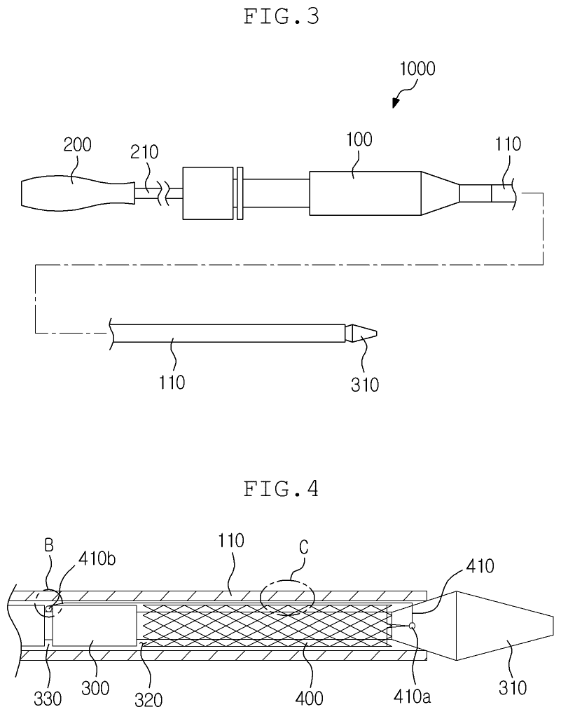

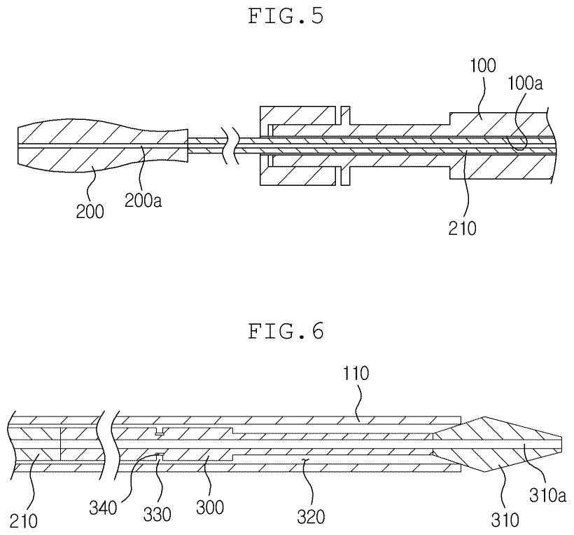

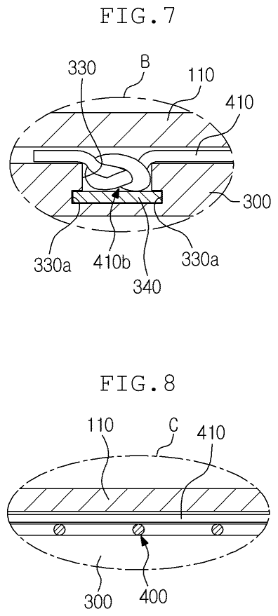

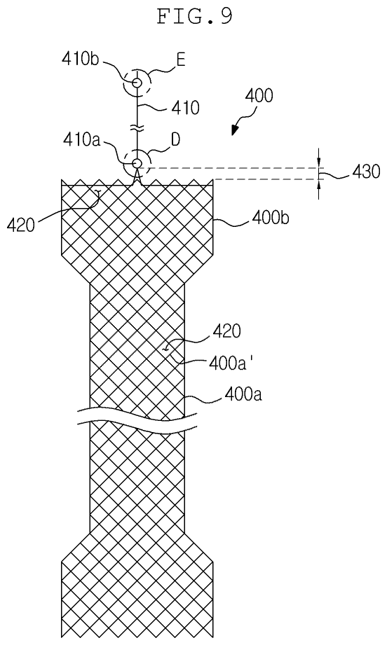

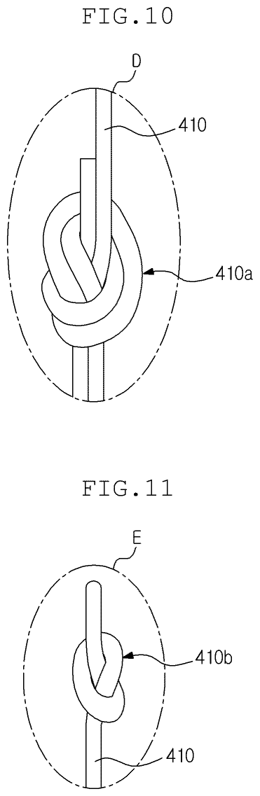

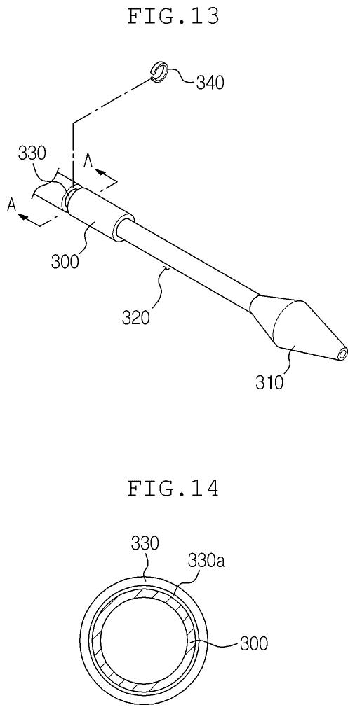

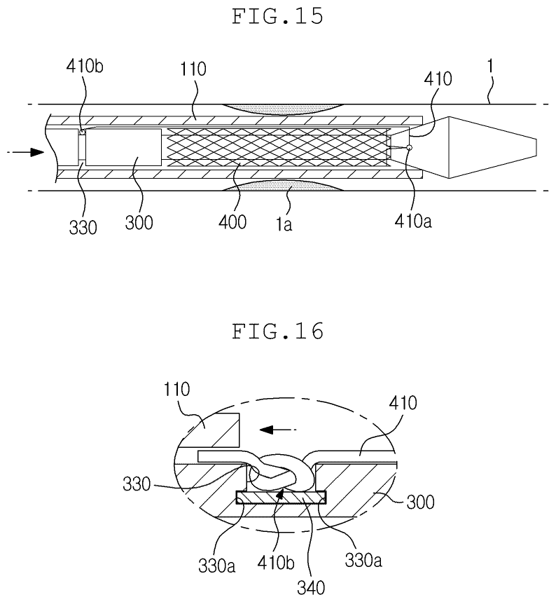

As shown in FIGS. 3 to 18, according to an embodiment of the present disclosure, a stent delivery system 1000 using a stent and an inner catheter is used to implant a stent 400 into a lumen 1 that is narrowed by a lesion area 1a when a stenotic or occlusive lesion area 1a occurs in a lumen 1 of the human body, such as the esophagus, duodenum, or biliary tract of the human body.

The stent delivery system 1000 includes an outer catheter 110, a first handle 100, a guide tube 210, a second handle 200, a guide tip 310, an inner catheter 300, a stent 400 with a traction thread 410, and a support ring 340.

The outer catheter 110 is connected to the first handle 100 and moved along the lumen 1 of the human body.

At this point, an inner passage 100a is formed through the inside space of the first handle 100 and communicates with the outer catheter 110. The outer catheter 110 is made of a soft tube.

The guide tube 210 is connected to the second handle 200 and movably inserted into the first handle 100 and the outer catheter 110.

At this point, the second handle 200 is located behind the first handle 100, and an inner passage 200a is formed through the inside space of the second handle 200 and communicates with the guide tube 210, and the guide tube 210 is made of a hard material such as steel and inserted into the inner passage 100a of the first handle 100 and into the outer catheter 110.

The inner catheter 300 is connected to the guide tube 210 and movably inserted into the outer catheter 110. The guide tip 310 is connected to one portion of the inner catheter 300 and caught by an opening of the outer catheter 110, and the portion of the inner catheter 300 includes a mounting space 320, in which the stent 400 with the traction thread 410 is compressed by the outer catheter 110 and mounted, close to the guide tip 31.

At this point, the inner catheter 300 is made of a soft tube and is connected to one portion of the guide tube 210 which is opposite to the second handle 200. the guide tip 310 is connected to one portion of the inner catheter 300 which is located opposite to the guide tube 210 and is caught by the opening of the outer catheter 110 to protrude outward of the outer catheter 110. An inner passage 310a is formed through the inside space of the guide tip 310 and communicates with the inner catheter 300.

Furthermore, a guide wire guides the inner catheter 300 and the outer catheter 110 to the lesion area 1a and penetrates the inner passage 200a of the second handle 200, the inside space of the guide tube 210, the inside space of the inner catheter 300, and the inner passage 310a of the guide tip 310.

The traction thread 410 is made up of a strand of nylon material. The traction thread 410 is woven around all space parts 420 closest to an end of the stent 400 in a circumferential direction of the stent 400 and then protrudes outward of the stent 400 with opposite parts of the traction thread 410 having different lengths.

The opposite parts having different lengths of the traction thread 410 are tied together so that a first knot part 410a is formed close to the end of the stent 400.

At this point, the first knot part 410a is formed by twisting the opposite parts of the traction thread 410 having different lengths together to form a loop shape and then inserting the opposite parts of the traction thread 410 having different lengths through the loop and tying up the loop, thereby located close to the end of the stent 400.

One part of the traction thread 410 protruding from the first knot part 410a is tied to form a second knot part 410b.

At this point, the second knot part 410b is formed by twisting a part of the traction thread 410 of a long length protruding from the first knot part 410a to form a loop shape and then inserting the part the traction thread 410 of a long length into the loop and tying up the loop.

Therefore, the second knot part 410b is formed a part of the traction thread 410 opposite to the first knot part 410a and located further away from the end of the stent 400 than the first knot part 410a.

In other words, the second knot part 410b is formed in the traction thread 410 to be located behind the mounting space 320 at the inner catheter 300, when the stent 400 is mounted to the mounting space 320.

The inner catheter 300 includes a ring-shaped first inserting groove 330 formed behind the mounting space 320, and the second knot part 410b is inserted into the first inserting groove 330.

At this point, the second knot part 410b is inserted into the first inserting groove 330 not to be exposed from the inner catheter 300.

The support ring 340 is inserted into the first inserting groove 330.

At this point, the support ring 340 has a ‘C’ shape with an opening at one side so as to be inserted into the first inserting groove 330 and is inserted into the first inserting groove 330 before inserting of the second knot part 410b.

The first inserting groove 330 has second inserting grooves 330a into which both side portions of the support ring 340 are inserted.

At this point, the second inserting grooves 330a are formed at the deepest side portions of the first inserting groove 330.

A distance 430 is formed between the end of the stent 400 and the first knot part 410a.

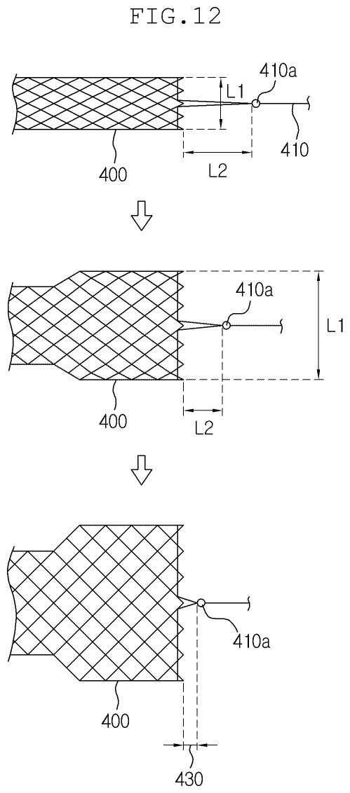

As shown in FIG. 12, describing in detail a reason of forming the distance 430, when the stent 400 of the present disclosure is compressed by the outer catheter 110 and mounted into the mounting space 320 of the inner catheter 300, a diametral length L1 of the stent 400 is formed shorter than a length L2 between the end of the stent 400 and the first knot part 410a, and when the stent 400 of the present disclosure is placed into the lesion area 1a, the diametral length L1 of the stent 400 is formed longer than the length L2 between the end of the stent 400 and the first knot part 410a. At this point, in order to prevent an expanded opening of the stent 400, i.e., an opening of the stent 400, from being caught by the first knot part 410a, the distance 430 is formed between the end of the stent 400 and the first knot part 410a.

At this point, the distance 430 may be formed to range from 3 mm to 10 mm.

The stent 400 is formed such that the wire 400a′ made from a shape memory alloy is woven or intersected another wire into a hollow cylindrical mesh shape, and includes a cylindrical stent 400a including a plurality of space parts 420 between the wires 400a′ and an extension tube part 400b formed on each portion of the cylindrical stent 400a and having a diameter greater than a diameter of the cylindrical stent 400a.

At this point, the traction thread 410 is fixed to an end of the extension tube part 400b, and the stent 400 may include PTFE and silicone films to close the space parts 420.

According to the embodiment of the present disclosure, the operation and effect of the stent 400 configured as described above and the stent delivery system 1000 to which the inner catheter 300 is used will be described as follows.

As shown in FIGS. 3 to 18, according to the embodiment of the present disclosure, the stent delivery system 1000 is configured with the stent 400 compressed by the outer catheter 110 and mounted to the mounting space 320 of the inner catheter 300.

Then, the traction thread 410 is located and overlapped between the outer catheter 110 and the stent 400 and between the outer catheter 110 and the inner catheter 300, and the thickness of the portion of the outer catheter 110 where the traction thread 410 is overlapped is prevented from becoming thicker due to a single strand of the traction thread 410.

Of course, even when the traction thread 410 is located in the inside space rather than the outside space of the stent 400, the traction thread 410 is made up of a single strand to prevent the thickness of a portion of the outer catheter 110 where the traction thread 410 is overlapped from becoming thicker.

Furthermore, the second knot part 410b of the traction thread 410 is inserted into the first inserting groove 330 not to be exposed from the inner catheter 300, thereby preventing the second knot part 410b from coming into contact with an inner surface of the outer catheter 110.
when the stenotic or occlusive lesion area 1a occurs in the lumen 1 of the human body, such as the esophagus, duodenum, biliary tract of the human body, the guide wire passes through the location of the lesion area 1a is confirmed endoscopically, and then the second handle 200, the guide tube 210, the inner catheter 300, and the guide tip 310 in order so that the guide wire is advanced to the lesion area 1a.

Furthermore, the first and second handles 100 and 200 of the stent delivery system 1000 are moved forward along the guide wire so that the outer catheter 110 and the inner catheter 300 are moved forward.

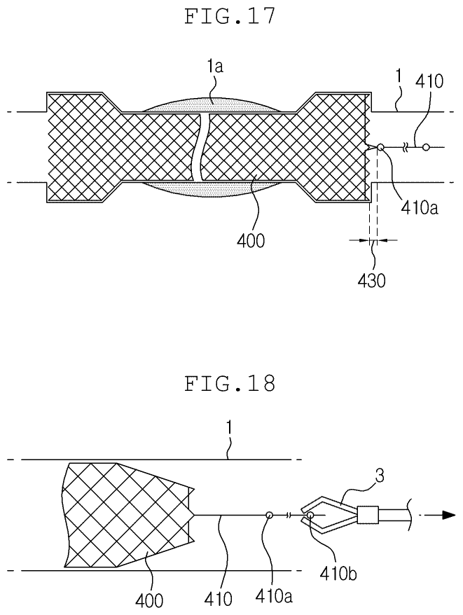

Then, as shown in FIG. 15, since the thickness of the outer catheter 110 is prevented from becoming thicker due to a single strand of the traction thread 410, the outer catheter 110 can be easily moved to the lesion area 1a with reduced catching by the lumen 1, i.e., reduced resistance during moving, and the outer catheter 110 can easily pass through the narrow lesion area 1a with reduced catching occurring when passing through the narrow the lesion area 1a, i.e., reduced resistance.

At this point, the thickness of a portion of the inner catheter 300 where the first inserting groove 330 becomes thinner due to the first inserting groove 330, and the outer catheter 110 and the guide tip 310 could be bent when caught by the lumen 1 and the lesion area 1a.

However, the portion of the inner catheter 300 with the first inserting groove 330 is supported by the support ring 340 inserted in the first and second inserting grooves 330 and 330a, and the inner catheter 300 is prevented from being bent when the outer catheter 110 and the guide tip 310 are caught by the lumen 1 and the lesion area 1a.

As shown in FIG. 16, the second knot part 410b inserted in the first inserting groove 330 barely protrudes from the inner catheter 300 not to come into contact with the inner surface of the outer catheter 110. Therefore, when the outer catheter 110 is pulled with the first handle 100, the second knot part 410b is not caught to facilitate pulling the outer catheter 110.

At this point, the first handle 100 and the outer catheter 110 are moved along the guide tube 210 toward the second handle 200.

Therefore, until the second knot part 410b of the traction thread 410 is separated from the first inserting groove 330 of the inner catheter 300, the outer catheter 110 is pulled.

Then, as shown in FIG. 17, the stent 400 of the present disclosure compressed by the outer catheter 110 expands promptly, and the second knot part 410b of the traction thread 410 is removed from the first inserting groove 330 of the inner catheter 300 by elasticity of the stent 400 that is recovered to an original state.

At this point, the distance 430 is formed between the end of the stent 400 and the first knot part 410a close thereto, so that the enlarged opening of the stent 400 is not caught by the first knot part 410a.

For this reason, the stent 400 of the present disclosure easily expands to widen the lesion area 1a.

As shown in FIG. 18, when a certain amount of time has passed after the stent 400 is placed into the stenotic or occlusive lesion area 1a occurring in the lumen 1, the stent 400 should be removed. At this point, the second knot part 410b of the traction thread 410 is pulled out with a pincer tool 3, such as forceps.

Then, the pulled second knot part 410b pulls the first knot part 410a so that the opening of the stent 400 is closed, and then the stent 400 may be easily removed from the lumen 1.

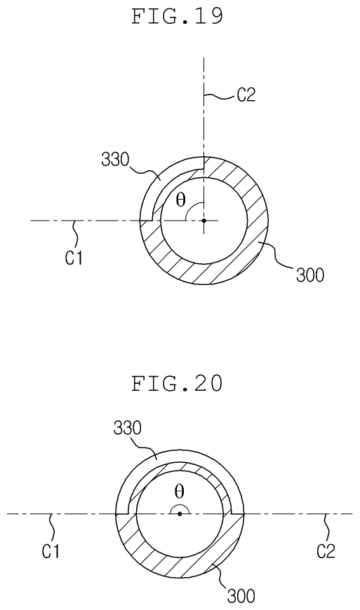

As shown in FIGS. 19 and 20, according to a modified example of the embodiment of the present disclosure, the first inserting groove 330 of the inner catheter 300 has a long shape formed along the circumference of the inner catheter 300 rather than a ring shape.

Furthermore, an angle θ between opposite ends of the first inserting groove 330 in the circumferential direction of the inner catheter 300 is formed to range from 90° to 180°.

In other words, the angle θ between a first imaginary line C1 passing through one end of the first inserting groove 330 and a central point of the inner catheter 300 and a second imaginary line C2 passing through the other end of the first inserting groove 330 and the central point of the inner catheter 300 is formed to range from 90° to 180°.

In other words, the first inserting groove 330 is formed on an outer surface of the inner catheter 300 by ¼ or ½ of the length of the circumference of the inner catheter 300.

Describing in detail a reason of forming the angle θ between the opposite ends of the first inserting groove 330 between 90° and 180°, when the angle θ between the opposite ends of the first inserting groove 330 is formed equal to or less than 90°, there is a concern that the second knot part 410b may not be properly inserted into the first inserting groove 330, and the angle θ between the opposite ends of the first inserting groove 330 is formed equal to or greater than 180°, a force that a portion of the inner catheter 300 where the first inserting groove 330 withstands is weak, and may be bent by being caught by the lumen 1 and the lesion area 1a, so the angle θ between the opposite ends of the first inserting groove 330 is formed to range from 90° to 180°.

Although the specific embodiments of the present disclosure have been provided, the present disclosure is not limited to the above-described embodiments, and various modifications, equivalents, additions and substitutions are possible by one of ordinary skill in the art to which this present disclosure belongs, without departing from the scope and spirit of the present disclosure.

What is claimed is:

1. A stent (400) with a traction thread (410), wherein the traction thread (410) is made up of a single strand, and the traction thread (410) is woven around all space parts (420) closest to an end of the stent (400), and then protrudes outwards of the stent (400) at opposite parts of the traction thread (410) having different lengths, and
the opposite parts having different lengths of the traction thread (410) are tied together with a first knot part (410a) formed close to the end of the stent (400), and one end of the traction thread (410) protruding from the first knot part (410a) is tied to form a second knot part (410b).

2. The stent of claim 1, wherein a distance (430) is formed between one end of the stent (400) and the first knot part 410a.

3. An inner catheter (300) movably inserted into an outer catheter (110) of a stent delivery system (1000), and connected to a guide tip (310) caught by an opening of the outer catheter (110) at one portion thereof, and including a mounting space (320) formed close to the guide tip (310), in which a stent (400) with a traction thread (410) at one portion thereof is compressed by the outer catheter 110 and mounted,
wherein one portion of the traction thread (410) protruding from the stent (400) is tied to form a second knot part (410b), and
a ring-shaped first inserting groove (330) is formed behind the mounting space (320) of the inner catheter (300) so that the second knot part 410b is inserted into the first inserting groove (330).

4. The inner catheter of claim 3, wherein a support ring (340) is inserted in the first inserting groove 330.

5. The inner catheter of claim 4, wherein the first inserting groove (330) includes second inserting grooves (330a) into which both side portions of the support ring (340) are inserted.

6. The inner catheter of claim 3, wherein the first inserting groove (330) has in a long shape formed along a circumference of the inner catheter (300) rather than a ring shape.

7. The inner catheter of claim 6, wherein in a circumferential direction of the inner catheter (300), an angle (θ) between opposite ends of the first inserting groove 330 is formed to range from 90° to 180°.

8. A stent delivery system comprising:
a first handle (100) connected to an outer catheter (110) moved along a lumen (1) of human body,
a second handle (200) connected to a guide tube (210) movably inserted into the first handle (100) and the outer catheter (110), and
an inner catheter (300), which is connected to the guide tube (210) and movably inserted into the outer catheter (110) and includes one portion connected to a guide tip (310) caught by an opening of the outer catheter (110), and, at the portion, includes a mounting space (320), in which a stent (400) with a traction thread (410) is compressed by the outer catheter (110) and mounted, formed close to the guide tip (310), wherein the stent (400) is placed into the lesion area (1a) in order to expand a stenotic or occlusive lesion area (1a) occurring in the lumen (1) and maintain a passage in the lumen (1),
wherein the traction thread (410) is made up of a single strand, and the traction thread (410) is woven around all space parts (420) closest to an end of the stent (400), and then protrudes outwards of the stent (400) at opposite parts of the traction thread (410) having different lengths,
the opposite parts having different lengths of the traction thread (410) are tied together to form a first knot part (410a) closest to one end of the stent (400), and one end of the traction thread (410) protruding from the first knot part (410a) is tied to form a second knot part (410b), and
a ring-shaped first inserting groove (330) is formed behind the mounting space (320) of the inner catheter (300) so that the second knot part (410b) is inserted into the first inserting groove (330).

9. The stent delivery system of claim 8, wherein a support ring (340) is inserted into the first inserting groove (330).

10. The stent delivery system of claim 9, wherein the first inserting groove (330) includes second inserting grooves (330a) into which both side portions of the support ring (340) are inserted.

11. The stent delivery system of claim 8, wherein a distance (430) is formed between one end of the stent (400) and the first knot part (410a).

12. The stent delivery system of claim 8, wherein the first inserting groove (330) has a long shape formed along a circumference of the inner catheter (300) rather than a ring shape.

13. The stent delivery system of claim 12, wherein in a circumferential direction of the inner catheter (300), an angle (θ) between opposite ends of the first inserting groove (330) is formed to range from 90° to 180°.
